(19) United States
(12) Patent Application Publication
(10) Pub. No.: US 2009/0131714 A1
(43) Pub. Date: May 21, 2009

(54) SYNTHESIS OF BETULONIC AND BETULINIC ALDEHYDES

(76) Inventors: Pavel A. Krasutsky, Duluth, MN (US); Kalyan Munshi, Duluth, MN (US)

Correspondence Address: SCHWEGMAN, LUNDBERG & WOESSNER, P.A., P.O. BOX 2938, MINNEAPOLIS, MN, 55402, US

(21) Appl. No.: 11/917,105

(22) PCT Filed: Jun. 7, 2006
(86) PCT No.: PCT/US2006/022177

§ 371 Date: Aug. 20, 2008

Related U.S. Application Data
(60) Provisional application No. 60/688,498, filed on Jun. 8, 2005.

(51) Int. Cl. C07C 51/16; C07C 45/00

(52) U.S. Cl. 562/498; 568/347

(57) ABSTRACT

The present invention provides for methods of selectively converting betulin to betulonic aldehyde. The present invention also provides for methods of selectively converting 3-substituted triterpen-28-ols to the corresponding 3-substituted triterpen-28-carboxaldehydes. Additionally, the present invention provides for methods of preparing betulonic aldehyde, betulonic acid, betulinic acid, and corresponding 3-substituted triterpenes.

### BACKGROUND OF THE INVENTION

Currently, there is a need for methods of preparing triterpenes such as betulonic aldehyde, betulonic acid, and betulinic acid. Additionally, there is a need for methods of selectively converting 3-substituted triterpen-28-ols to the corresponding 3-substituted triterpen-28-carboxaldehydes. Such methods would employ relatively inexpensive, nontoxic and environmentally safe reagents and solvents, compared to known methods.

### SUMMARY OF THE INVENTION

Provided herein is a method for preparing a compound of formula (I):

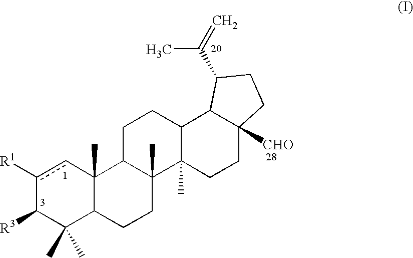

wherein

R¹ is hydrogen or hydroxy;

R³ is acyloxy or oxo (═O);

the method comprising contacting a metal alcoholate, a compound of the formula (A):

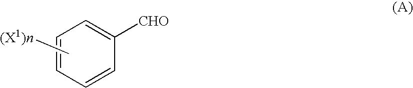

wherein,

each X¹ is independently halo, nitro, hydroxyl, (C₁-C₆)alkyl, (C₁-C₆)alkoxy, COOR⁷, or NR¹⁶R¹⁷, wherein each of R¹⁶ and R¹⁷ are independently H or (C₁-C₆)alkyl; and

n is 0, 1, 2, 3, 4 or 5;

and a compound of formula (II):

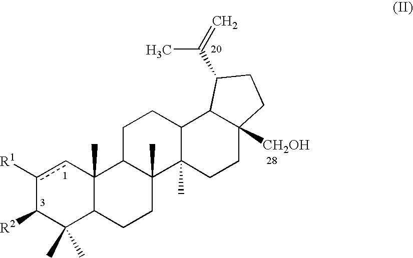

wherein,

R² is hydroxyl or acyloxy; for a period of time effective to provide the compound of formula (I).

A specific value of R¹ is hydrogen.

In one embodiment, R³ is acyloxy. In a specific embodiment, R³ is acetoxy. In another embodiment, R³ is oxo (═O).

A specific value of X¹ is chlorine. Another specific value of X¹ is nitro.

In one embodiment, n can be 0. In another embodiment, n can be 1. Alternatively, n can be 2, 3, 4, or 5.

The compound of formula (A) can be 2-chlorobenzaldehyde or 2-nitrobenzaldehyde.

The metal alcoholate can be an aluminum alcoholate. The metal alcoholate can also be aluminum iso-propoxide [Al(i-OPr)₃].

A specific value of R² is hydroxyl. Another value of R² is acyloxy. Another specific value of R² is acetoxy (CH₃C(═O)O).

The contacting of the compound of formula II and the compound of formula A can occur in the presence of a solvent. The solvent can be a polar non-protic solvent. The contacting can occur at a temperature of at least about 10° C., at least about 20° C., or at least about 40° C.

The yield of the compound of formula I can be at least about 50, at least about 60, or at least about 70 molar percent. The compound of formula I can have a purity of at least about 70 percent, at least about 90 percent, or at least about 95 percent, as determined by HPLC. The compound of formula I can be optionally further purified.

The metal alcoholate can be employed in at least about 1 molar equivalents, at least about 2 molar equivalents, at least about 4 molar equivalents, at least about 6 molar equivalents, in relation to the compound of formula (II). The contacting can occur for at least about 1 hour, at least about 2 hours, or at least about 5 hours.

The compound of formula (II) can be employed in at least about 0.5 kilogram, at least about 1 kilogram, or at least about 10 kilograms. At least about 0.5 kilograms of the compound of formula (I) can be obtained. In other embodiments, at least about 1 kilogram, at least about 2 kilograms, or at least about 10 kilograms of the compound of formula (I) can be obtained.

In another embodiment of the invention, the compound of formula (I) can be contacted the with an effective amount of an alkali metal chlorite, for a period of time effective to provide a compound of formula (III):

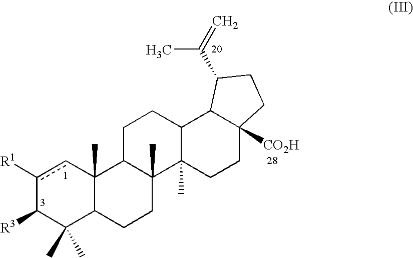

or a salt thereto,
wherein

R¹ is hydrogen or hydroxy; and

R³ is acyloxy or oxo (═O).

The alkali metal chlorite can be NaClO₂, KClO₂, or a combination thereof.

The compound of formula (I) can be contacted with about 5 molar equivalents to about 10 molar equivalents of the alkali metal chlorite, relative to the compound of formula (I). The compound of formula (I) can also be contacted with about 2 molar equivalents to about 5 molar equivalents of the alkali metal chlorite, relative to the compound of formula (I).

The contacting can be carried out at a temperature of about 10° C. to about 120° C.

The contacting can be carried out for a period of time of about 30 minutes to about 48 hours.

The contacting can be carried out in a solvent system selected from water, an alcohol, unsaturated hydrocarbons, mineral oil, ether, dioxane, DMF, DMA, DMSO, benzene, toluene, xylene, pyridine, chloroform, methylene chloride, morpholine, N-methylmorpholine, cyclohexane, cyclohexanone, acetone, ethyl acetate, pyrrole, and pyrrolidone, or a combination thereof.

In one embodiment, at least about 0.5 kg of the compound of formula (III) can be obtained. In another embodiment, at least about 1 kg of the compound of formula (III) can be obtained.

In one embodiment, at least about 70 mol % of the compound of formula (III) can be obtained, based upon the compound of formula (I). In another embodiment, at least about 85 mol %, or about 90 mol % of the compound of formula (III) can be obtained, based upon the compound of formula (I).

The compound of formula (III) can optionally be purified. The purifying can include washing the compound of formula (III). In another embodiment, the purifying can include recrystallizing the compound of formula (III). In yet another embodiment, the purifying can include separating the compound of formula (III) from any unreacted triterpene compound by converting the compound of formula (III) into a carboxylic acid salt and separating the carboxylic acid salt from the unreacted triterpene compound. The carboxylic acid salt can include a Li, Na, K, Mg, Ca, Sr, Ba, or Al cation. Alternatively, the carboxylic acid salt can include a nitrogen cation, such as an ammonium cation.

The compound of formula (III) can be obtained having a purity of at least about 80 wt. %. The compound of formula (III) can also be obtained having a purity of at least about 95 wt. %.

The method of preparing a compound of formula (III) can further include employing a free halogen scavenger. The halogen scavenger that can be an unsaturated hydrocarbon. In another embodiment, the halogen scavenger can be selected from the group of amylene, cyclohexene, methylcyclohexene and cyclopentene.

In another embodiment of the invention, the compound of formula (III) can be contacted with a metal alcoholate for a period of time effective to provide a compound of formula (IV):

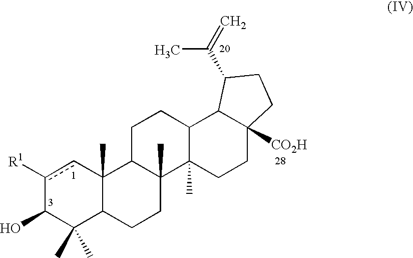

or a salt thereof,

wherein R¹ is hydrogen or hydroxy.

The bond between carbons 1 and 2 can be a single bond. In another embodiment, the bond between carbons 1 and 2 can be a double bond.

The compound of formula (III) can be betulonic acid. In another embodiment, R³ of the compound of formula (III) can be acyloxy. In a specific embodiment, R³ of the compound of formula (III) is acetoxy.

The metal alcoholate can be aluminum iso-propoxide.

The contacting can occur in the presence of a compound of formula (B):

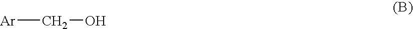

wherein Ar is aryl or heteroaryl.

Alternatively, the contacting can occur in the presence of a compound of the formula (C):

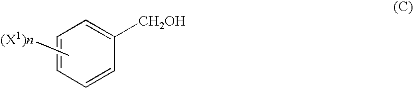

wherein,

each X¹ is independently halo, nitro, hydroxyl, (C₁-C₆)alkyl, (C₁-C₆)alkoxy, COOR⁷, or NR¹⁶R¹⁷, wherein each R¹⁶ and R¹⁷ is independently H or (C₁-C₆)alkyl; and

n is 0, 1, 2, 3, 4 or 5.

In one embodiment, n is 0. In another embodiment, n is 1.

In one specific embodiment, the compound of formula (C) is benzyl alcohol.

In another embodiment, the contacting can occur in the presence of a compound of the formula (D):

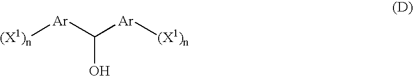

wherein each Ar is independently aryl or heteroaryl;

each X¹ is independently halo, nitro, hydroxyl, (C₁-C₆)alkyl, (C₁-C₆)alkoxy, COOR¹⁷, or NR⁶R⁷,

each R¹⁶ and R¹⁷ is independently H or (C₁-C₆)alkyl; and

each n is independently 0, 1, 2, 3, 4 or 5.

In certain embodiments, each n can be 0. In other embodiments, n can be 1. In other embodiments, each n can be different and can be any value from 0 to 5, inclusive.

In one embodiments, compound of formula (D) is a compound of formula (D-1):

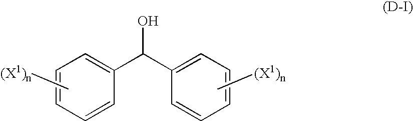

wherein X¹ and n are as defined above.

The contacting can occur in the presence of a solvent. The contacting can occur in the presence of a solvent selected from the group of ethyl ether, tetrahydrofuran (THF), dioxane, acetonitrile, dimethylformamide (DMF), dimethylacetamide (DMA), ethyl acetate, or a combination thereof.

The contacting can occur at a temperature of at least about 50° C.

The compound of formula (IV) can be purified. The purifying the compound of formula (IV) can be performed by washing the compound of formula (IV). The purifying the compound of formula (IV) can be performed by washing the compound of formula (IV) with an aqueous acid, an aqueous base, a non-polar aprotic solvent, a polar aprotic solvent, or a mixture thereof.

The compound of formula (IV) can be provided in a yield of at least 95 molar percent.

The compound of formula (IV) can be provided with a purity of at least 95 percent, as determined by HPLC.

The metal alcoholate can be employed in at least about 2 molar equivalents, in relation to the compound of formula (III). Alternatively, the metal alcoholate can be employed in at least about 4 molar equivalents, in relation to the compound of formula (III). The contacting can occur for at least about 2 hours.

The compound of formula (III) can be employed in at least about 1 kilogram. At least about 1 kilogram of the compound of formula (IV) can be obtained.

In another embodiment of the invention, a method is provided for preparing a compound of formula (IV):

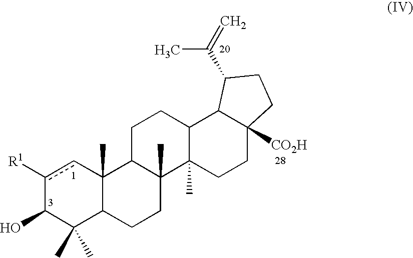

or a salt thereof,

wherein

R¹ is hydrogen or hydroxy; and

the bond shown as ----- is present or absent; the method comprising the steps of:

(1) contacting a metal alcoholate, a compound of the formula (A):

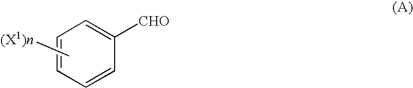

wherein,

each X¹ is independently halo, nitro, hydroxyl, (C₁-C₆)alkyl, (C₁-C₆)alkoxy, COOR⁷, or NR¹⁶R¹⁷, wherein each of R¹⁶ and R¹⁷ are independently H or (C₁-C₆)alkyl; and

n is 0, 1, 2, 3, 4 or 5;

and a compound of formula (II):

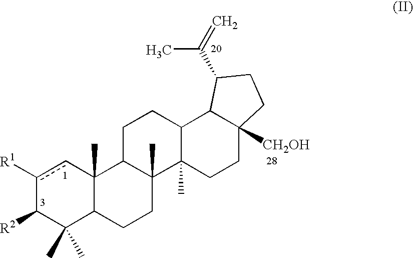

wherein,

R² is hydroxyl or acyloxy;

for a period of time effective to provide the compound of formula (I):

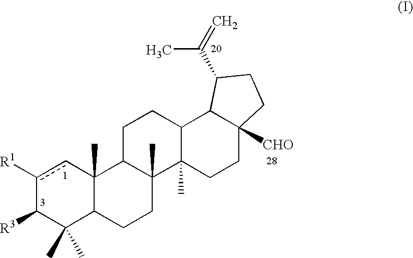

wherein

R² is hydrogen or hydroxy;

R³ is acyloxy or oxo (═O);

(2) contacting the compound of formula (I) with an effective amount of an alkali metal chlorite, for a period of time effective to provide a compound of formula (III):

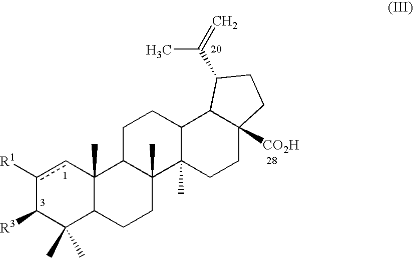

or a salt thereof,
wherein

R¹ is hydrogen or hydroxy;

R³ is acyloxy or oxo (═O); and

(3) contacting a metal alcoholate and the compound of formula (III) for a period of time effective to provide the compound of formula (IV).

### DETAILED DESCRIPTION OF THE INVENTION

As used herein, the following terms and expressions have the indicated meanings. It will be appreciated that the methods of the present invention can employ and/or provide compounds that can contain asymmetrically substituted carbon atoms, and can be isolated in optically active or racemic forms. It is well known in the art how to prepare optically active forms, such as by resolution of racemic forms or by synthesis, from optically active starting materials.

All chiral, diastereomeric, racemic forms and all geometric isomeric forms of a structure are intended, unless the specific stereochemistry or isomeric form is specifically indicated. The processes to prepare or manufacture compounds useful in the present invention are contemplated to be practiced on at least a multigram scale, kilogram scale, multikilogram scale, or industrial scale. Multigram scale, as used herein, is preferably the scale wherein at least one starting material is present in 10 grams or more, more preferably at least 50 grams or more, even more preferably at least 100 grams or more. Multi-kilogram scale, as used herein, is intended to mean the scale wherein more than one kilogram of at least one starting material is used. Industrial scale as used herein is intended to mean a scale which is other than a laboratory scale and which is sufficient to supply product sufficient for either clinical tests or distribution to consumers.

One diastereomer of a compound disclosed herein may display superior activity compared with the other. When required, separation of the racemic material can be achieved by HPLC using a chiral column or by a resolution using a resolving agent such as camphonic chloride as in Tucker, et al., J. Med. Chem. 37:2437 (1994). A chiral compound described herein may also be directly synthesized using a chiral catalyst or a chiral ligand, e.g. Huffman, et al., J. Org. Chem., 60:1590 (1995).

The present invention is intended to include all isotopes of atoms occurring on the compounds useful in the present invention. Isotopes include those atoms having the same atomic number but different mass numbers. By way of general example and without limitation, isotopes of hydrogen include tritium and deuterium. Isotopes of carbon include C-13 (¹³C) and C-14 (¹⁴C).

### DEFINITIONS

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the compounds useful in the present invention can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., p. 1418 (1985), the disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

“Stable compound” and “stable structure” are meant to indicate a compound that is sufficiently robust to survive isolation to a useful degree of purity from a reaction mixture, and formulation into an efficacious therapeutic agent. Only stable compounds are contemplated by the present invention.

“Substituted” is intended to indicate that one or more hydrogens on the atom indicated in the expression using “substituted” is replaced with a selection from the indicated group(s), provided that the indicated atom's normal valency is not exceeded, and that the substitution results in a stable compound. Suitable indicated groups include, e.g., alkyl, alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano. When a substituent is keto (i.e., ═O) or thioxo (i.e., ═S) group, then 2 hydrogens on the atom are replaced.

The term “alkyl” refers to a monoradical branched or unbranched saturated hydrocarbon chain preferably having from 1 to 40 carbon atoms, more preferably 1 to 30 carbon atoms, and even more preferably 1 to 26 carbon atoms. This term is exemplified by groups such as methyl, ethyl, n-propyl, iso-propyl, n-butyl, iso-butyl, sec-butyl, n-hexyl, n-decyl, tetradecyl, stearyl, octyl, decyl, lauryl, myristyl, palmityl, and the like.

The alkyl can optionally be substituted with one or more alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl, cyano, NRₓRₓ or COORₓ, wherein each Rₓ is independently H or alkyl.

The alkyl can optionally be interrupted with one or more non-peroxide oxy (—O—), thio (—S—), sulfonyl (SO), or sulfoxide (SO₂) groups.

The alkyl can optionally be at least partially unsaturated, thereby providing an alkenyl or alkynyl.

The term “alkoxy” refers to the groups alkyl-O—, where alkyl is defined herein. Preferred alkoxy groups include, e.g., methoxy, ethoxy, n-propoxy, iso-propoxy, n-butoxy, tert-butoxy, sec-butoxy, n-pentoxy, n-hexoxy, 1,2-dimethylbutoxy, and the like.

The alkoxy can optionally be substituted with one or more alkyl, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano.

The term “aryl” refers to an unsaturated aromatic carbocyclic group of from 6 to 20 carbon atoms having a single ring (e.g., phenyl) or multiple condensed (fused) rings, wherein at least one ring is aromatic (e.g., naphthyl, dihydrophenanthrenyl, fluorenyl, or anthryl). Preferred aryls include phenyl, naphthyl and the like.

The aryl can optionally be substituted with one or more alkyl, alkoxy, halo, haloalkyl, hydroxy, hydroxyalkyl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano.

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

The cycloalkyl can optionally be substituted with one or more alkyl, alkoxy, halo, haloalkyl, hydroxy, hydroxyalkyl, aryl, heteroaryl, heterocycle, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano.

The cycloalkyl can optionally be at least partially unsaturated, thereby providing a cycloalkenyl.

The term “halo” refers to fluoro, chloro, bromo, and iodo. Similarly, the term “halogen” refers to fluorine, chlorine, bromine, and iodine.

The term “haloalkyl” refers to alkyl as defined herein substituted by 1 or more halo groups as defined herein, which may be the same or different. In one embodiment, the haloalkyl can be substituted with 1, 2, 3, 4, or 5 halo groups. In another embodiment, the haloalkyl can by substituted with 1, 2, or 3 halo groups. Representative haloalkyl groups include, by way of example, trifluoromethyl, 3-fluorododecyl, 12,12,12-trifluorododecyl, 2-bromooctyl, 3-bromo-6-chloroheptyl, 1H, 1H-perfluorooctyl, and the like.

The term “heteroaryl” is defined herein as a monocyclic, bicyclic, or tricyclic ring system containing one, two, or three aromatic rings and containing at least one nitrogen, oxygen, or sulfur atom in an aromatic ring, and which can be unsubstituted or substituted, for example, with one or more, and in particular one to three, substituents, like halo, alkyl, hydroxy, hydroxyalkyl, alkoxy, alkoxyalkyl, haloalkyl, nitro, amino, alkylamino, acylamino, alkylthio, alkylsulfinyl, and alkylsulfonyl. Examples of heteroaryl groups include, but are not limited to, 2H-pyrrolyl, 3H-indolyl, 4H-quinolizinyl, 4nH-carbazolyl, acridinyl, benzo[b]thienyl, benzothiazolyl, β-carbolinyl, carbazolyl, chromenyl, cinnaolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, naptho[2,3-b], oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrimidinyl, pyrrolyl, quinazolinyl, quinolyl, quinoxalinyl, thiadiazolyl, thianthrenyl, thiazolyl, thienyl, triazolyl, and xanthenyl. In one embodiment the term “heteroaryl” denotes a monocyclic aromatic ring containing five or six ring atoms containing carbon and 1, 2, 3, or 4 heteroatoms independently selected from the group non-peroxide oxygen, sulfur, and N(Z) wherein Z is absent or is H, O, alkyl, phenyl or benzyl. In another embodiment heteroaryl denotes an ortho-fused bicyclic heterocycle of about eight to ten ring atoms derived therefrom, particularly a benz-derivative or one derived by fusing a propylene, or tetramethylene diradical thereto.

The heteroaryl can optionally be substituted with one or more alkyl, alkoxy, halo, haloalkyl, hydroxy, hydroxyalkyl, aryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano.

The term “heterocycle” refers to a saturated or partially unsaturated ring system, containing at least one heteroatom selected from the group oxygen, nitrogen, and sulfur, and optionally substituted with alkyl or C(═O)OR^{b}, wherein Rb is hydrogen or alkyl. Typically heterocycle is a monocyclic, bicyclic, or tricyclic group containing one or more heteroatoms selected from the group oxygen, nitrogen, and sulfur. A heterocycle group also can contain an oxo group (═O) attached to the ring. Non-limiting examples of heterocycle groups include 1,3-dihydrobenzofuran, 1,3-dioxolane, 1,4-dioxane, 1,4-dithiane, 2H-pyran, 2-pyrazoline, 4H-pyran, chromanyl, imidazolidinyl, imidazolinyl, indolinyl, isochromanyl, isoindolinyl, morpholine, piperazinyl, piperidine, piperidyl, pyrazolidine, pyrazolidinyl, pyrazolinyl, pyrrolidine, pyrroline, quinuclidine, and thiomorpholine.

The heterocycle can optionally be substituted with one or more alkyl, alkoxy, halo, haloalkyl, hydroxy, hydroxyalkyl, aryl, heteroaryl, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano.

Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles.

Another class of heterocyclics is known as “crown compounds” which refers to a specific class of heterocyclic compounds having one or more repeating units of the formula [—(CH₂—)ₐA-] where a is equal to or greater than 2, and A at each separate occurrence can be O, N, S or P. Examples of crown compounds include, by way of example only, [—(CH₂)₃—NH—]₃, [—((CH₂)₂—O)₄—((CH₂)₂—NH)₂] and the like. Typically such crown compounds can have from 4 to 10 heteroatoms and 8 to 40 carbon atoms.

The term “alkanoyl” refers to C(═O)R, wherein R is an alkyl group as previously defined.

The term “acyloxy” refers to —O—C(═O)R, wherein R is an alkyl group as previously defined. Examples of acyloxy groups include, but are not limited to, acetoxy, propanoyloxy, butanoyloxy, and pentanoyloxy. Any alkyl group as defined above can be used to form an acyloxy group.

The term “alkoxycarbonyl” refers to —C(═O)OR, wherein R is an alkyl group as previously defined.

The term “amino” refers to —NH₂, and the term “alkylamino” refers to —NR₂, wherein at least one R is alkyl and the second R is alkyl or hydrogen. The term “acylamino” refers to RC(═O)N, wherein R is alkyl or aryl.

The term “nitro” refers to —NO₂.

The term “trifluoromethyl” refers to —CF₃.

The term “trifluoromethoxy” refers to —OCF₃.

The term “cyano” refers to —CN.

The term “hydroxy” or “hydroxyl” refers to —OH.

The term “oxy” refers to —O—.

The term “thio” refers to —S—.

As to any of the above groups, which contain one or more substituents, it is understood, of course, that such groups do not contain any substitution or substitution patterns which are sterically impractical and/or synthetically non-feasible. In addition, the compounds of this invention include all stereochemical isomers arising from the substitution of these compounds.

Selected substituents within the compounds described herein are present to a recursive degree. In this context, “recursive substituent” means that a substituent may recite another instance of itself. Because of the recursive nature of such substituents, theoretically, a large number may be present in any given claim. One of ordinary skill in the art of medicinal chemistry understands that the total number of such substituents is reasonably limited by the desired properties of the compound intended. Such properties include, by of example and not limitation, physical properties such as molecular weight, solubility or log P, application properties such as activity against the intended target, and practical properties such as ease of synthesis.

Recursive substituents are an intended aspect of the invention. One of ordinary skill in the art of medicinal and organic chemistry understands the versatility of such substituents. To the degree that recursive substituents are present in an claim of the invention, the total number will be determined as set forth above.

As used herein, “triterpene” or “triterpenoid” refers to a plant secondary metabolite that includes a hydrocarbon, or its oxygenated analog, that is derived from squalene by a sequence of straightforward cyclizations, functionalizations, and sometimes rearrangement. Triterpenes or analogues thereof can be prepared by methods known in the art, i.e., using conventional synthetic techniques or by isolation from plants. Suitable exemplary triterpenes and the biological synthesis of the same are disclosed, e.g., in R. B. Herbert, The Biosynthesis of Secondary Plant Metabolites, 2nd ed. (London: Chapman 1989). The term “triterpene” refers to one of a class of compounds having approximately 30 carbon atoms and synthesized from six isoprene units in plants and other organisms. Triterpenes consist of carbon, hydrogen, and optionally oxygen. Most triterpenes are secondary metabolites in plants. Most, but not all, triterpenes are pentacyclic. Examples of triterpenes include betulin, allobetulin, lupeol, friedelin, and all sterols, including lanosterol, stigmasterol, cholesterol, β-sitosterol, and ergosterol.

The triterpenes used in the methods disclosed herein typically have “trans” ring junctions between each of the carbocyclic rings (A-E). Unless specifically otherwise noted, a hydrogen substituent at a ring junction opposite (one carbon atom away from) a methyl substituent at a ring junction will be trans to the methyl substituent, as would be readily understood by those of skill in the art upon viewing the structural drawings.

As used herein, “betulin” refers to 30,28-dihydroxy-lup-20(29)-ene. Betulin is a pentacyclic triterpenoid derived from the outer bark of paper birch trees (Betula papyrifera, B. pendula, B. verucosa, etc.). The CAS Registry No. is 473-98-3. It can be present at concentrations of up to about 24% of the bark of white birch. Merck Index, twelfth edition, page 1236 (1996). Structurally, betulin is shown below:

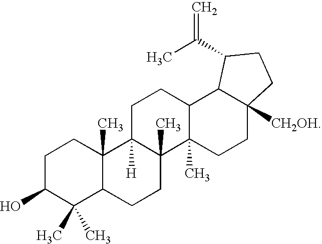

As used herein, “belulonic aldehyde” refers to a compound of the formula

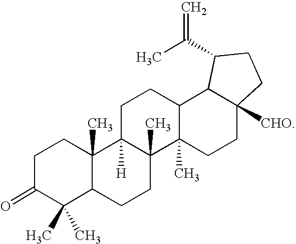

As used herein, “belulonic acid” refers to a compound of the formula

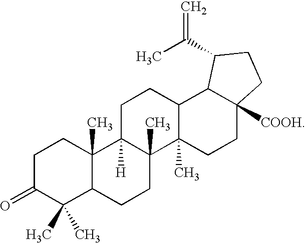

As used herein, “betulinic acid” refers to 3(β)-hydroxy-20(29)-lupaene-28-oic acid; 9-hydroxy-1-isopropenyl-5a,5b,8,8,11a-pentamethyl-eicosahydro-cyclopenta[a]chrysene-3a-carboxylic acid. The CAS Registry No. is 472-15-1. Structurally, betulinic acid is shown below:

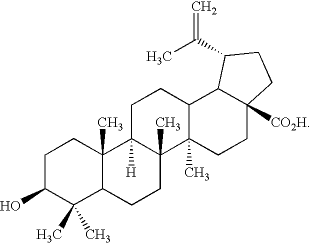

As used herein, “amino acid” refers to the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also comprises natural and unnatural amino acids bearing a conventional amino protecting group (e.g. acetyl or benzyloxycarbonyl), as well as natural and unnatural amino acids protected at the carboxy terminus (e.g. as a (C₁-C₆)alkyl, phenyl or benzyl ester or amide; or as an α-methylbenzyl amide). Other suitable amino and carboxy protecting groups are known to those skilled in the art (See for example, T. W. Greene, Protecting Groups In Organic Synthesis; Third Edition, Wiley: New York, 1999, and references cited therein). An amino acid can be linked to the remainder of a compound of formula (I)-(IV) through the carboxy terminus, the amino terminus, or through any other convenient point of attachment, such as, for example, through the sulfur of cysteine.

The term “peptide” describes a sequence of 2 to 25 amino acids (e.g. as defined hereinabove) or peptidyl residues. The sequence may be linear or cyclic. For example, a cyclic peptide can be prepared or may result from the formation of disulfide bridges between two cysteine residues in a sequence. A peptide can be linked to the remainder of a compound of formula (I)-(IV) through the carboxy terminus, the amino terminus, or through any other convenient point of attachment, such as, for example, through the sulfur of a cysteine. Preferably a peptide comprises 3 to 25, or 5 to 21 amino acids. Peptide derivatives can be prepared as disclosed in U.S. Pat. Nos. 4,612,302; 4,853,371; and 4,684,620.

The term “polyethyleneimine” refers to the group (—NHCH₂CH₂—)ₓ[—N(CH₂CH₂NH₂)CH₂CH₂—]_{y}. Polyethyleneimine can be attached to a compound through either of the nitrogen atoms marked with hash marks. “Poly(ethylene glycol)” refers to the compound H(OCH₂CH₂)nOH. It can be attached to a compound through its terminal hydroxyl.

The term “direct bond” refers to a group being absent.

As used herein, “metal alcoholate” or “alcoholate” refers to an organic alcohol wherein the hydroxy hydrogen has been replaced with a metal, e.g., (CH₃CH₂O)₃Al. Metal alcoholates are suitable reagents for triterpene purification because it is believed that metal alcoholates bind strongly and irreversibly to acids and tannins, therefore providing complete discoloration of the total extract. Suitable specific metal alcoholates include, e.g., sodium methoxide (NaOMe), sodium ethoxide (NaOEt), potassium methoxide (KOMe), potassium ethoxide (KOEt), aluminum iso-propoxide [Al(i-OPr)₃], aluminum tert-butoxide [Al(t-OBu)₃], and aluminum methoxide [Al(OMe)₃].

As used herein, “aluminum iso-propoxide” refers to a compound of the formula Al(i-OPr)₃.

As used herein, “contacting” refers to the act of touching, making contact, or bringing into immediate proximity.

As used herein, “washing” refers to the process of purifying a solid mass (e.g., crystals or an amorphous solid) by passing a liquid over and/or through the solid mass, as to remove soluble matter. The process includes passing a solvent, such as distilled water, over and/or through a precipitate obtained from filtering, decanting, or a combination thereof. For example, in one embodiment of the invention, washing includes contacting solids with water, vigorously stirring (e.g., for two hours), and filtering. The solvent can be water, can be an aqueous solvent system, or can be an organic solvent system. Additionally, the washing can be carried out with the solvent having any suitable temperature. For example, the washing can be carried out with the solvent having a temperature between about 0° C. and about 100° C.

As used herein, “stereoselective reduction,” “selectively converting a triterpen-3-one to the corresponding triterpen-3-ol” or “selectively reducing a triterpen-3-one to the corresponding triterpen-3-ol” refers to the conversion of the functional group at the C-3 position of a triterpene, e.g., reduction of the ketone to the corresponding beta (β) C-3 hydroxyl triterpene. In one embodiment, the ratio of beta (β) C-3 hydroxyl triterpene to alpha (α) C-3 hydroxyl triterpene is at least about 90:10. In another embodiment of the invention, the ratio of beta (β) C-3 hydroxyl triterpene to alpha (α) C-3 hydroxyl triterpene is at least about 95:5. In another embodiment of the invention, the ratio of beta (β) C-3 hydroxyl triterpene to alpha (α) C-3 hydroxyl triterpene is at least about 98:2. In another embodiment of the invention, the ratio of beta (β) C-3 hydroxyl triterpene to alpha (α) C-3 hydroxyl triterpene is at least about 99:1.

Any patent, patent document, or reference disclosed herein is incorporated into reference into this invention and forms part of this invention.

The following example is introduced in order that the invention may be more readily understood. It is intended to illustrate the invention but not limit its scope.

### EXAMPLES

### Example 1

### Oxidation of Betulin to Betulonic Aldehyde

In a 500 mL glass reactor 10 g Betulin (22.6 mmol) was dissolved in 200 mL THF, followed by the addition of 20 g Aluminum Isopropoxide (98.0 mmol) and 25 mL of 2-chlorobenzaldehyde (223.2 mmol). The resulting mixture was vigorously stirred at room temperature (25° C.) for 1 hour. The whole reaction mixture was poured down into a beaker containing 800 mL faintly acidified (2% HCl) cold water, constantly stirred for 15 minutes, allowed to settle for 1 hour, and filtered off. The residue was several times washed off (10×20 mL hexanes) and dried in air. It was then treated with 100 mL THF, homogenized in a homogenizer (8000 rpm, 5 minutes), filtered off, and finally the filtrate was evaporated in a rotor evaporator (50° C., 20 mbar). The resulting pale yellow, sticky, solid was recrystallized from 2-propanol (70 mL) to obtain 6.75 g (15.4 mmol) pale yellow, solid betulonic aldehyde of 95%⁺ purity. The conversion was approximately 100% and the overall yield after recrystallization was 68%.

### Example 2

### Oxidation of 3-β-Acetoxy Betulin to 3-O-Acetoxy Betulinic Aldehyde

In a 250 mL glass reactor 5 g 3-acetoxy betulin (10.3 mmol) was dissolved in 100 mL THF, followed by the addition of 5 g aluminum isopropoxide (24.5 mmol) and 6 g 2-nitrobenzaldehyde (39.7 mmol). The resulting mixture was refluxed (65° C.) with constant vigorous stirring for 2 hours. The whole reaction mixture was poured down into a beaker containing 800 mL of cold water, stirred for 10 minutes, and filtered off. To the dried residue 150 mL dichloromethane was added, homogenized in a homogenizer (8000 rpm, 5 minutes), filtered off (followed by washing the residue with 10×5 mL of CH₂Cl₂), and the filtrate was evaporated in a rotor evaporator (25° C., 350 mbar). To the resulting solid, 150 mL nitromethane was added, well homogenized (8000 rpm; 5 minutes), filtered off, and the residue was dried in a vacuum oven (50° C.; 350 mbar) for 24 hours. Finally the dried product (4.27 g) was recrystallized from 2-propanol (40 mL) to obtain 3.49 g (7.2 mmol) marble-white, solid 3-acetoxy betulinic aldehyde of 95%⁺ purity. The conversion was approximately 100% and overall yield after recrystallization was 70%.

### Example 3

### Oxidation of 3-O-(3′,3′-dimethylsuccinyl)betulinic aldehyde to 3-O-(3′,3′-dimethylsuccinyl)betulinic Acid

Sodium chlorite (1 g, 9 mmol) and potassium phosphate monobasic (1.22 g, 9 mmol) in water (35 mL) was added dropwise to a stirred mixture of 3-O-(3′,3′-dimethylsuccinyl)betulinic aldehyde (0.88 g, 1.5 mmol), 2-methyl-2-butene (15 mL) and tert-butanol (50 mL). The mixture was stirred for 16 hours at room temperature, diluted with water (100 mL) and diethyl ether (50 mL). The organic layer was separated, dried with sodium sulfate and evaporated in vacuo to give crude product. The crude product was recrystallized twice from hexane to provide 0.65 g of pure 3-O-(3′,3′-dimethylsuccinyl)betulinic acid (72% yield).

¹H NMR (pyridine-d5): 0.65-1.95 (complex CH—, CH₂, 22H) 0.73, 0.92, 0.97, 1.01, 1.05 (each 3H, s; 4-(CH₃)₂, 8-CH₃, 10-CH₃, 14-CH₃), 1.55 (6H, s, 3′-CH₃.times.2), 1.80 (3H, s, 20-CH.sub.3), 2.24 (2H, m), 2.67 (2H, m), 2.89, 2.94 (each 1H, d, J=15.5 Hz, H-2′), 3.53 (1H, m, H-19), 4.76 (1H, dd, J=5.0, 11.5 Hz, H-3), 4.78, 4.95 (each 1H, br s, H-30).

### Example 4

### Stereoselective Reduction of Betulonic Acid into 3-β-Betulinic Acid

In a 250 mL glass reactor 5 g of betulonic acid (11.0 mmol) was dissolved in 100 mL THF followed by the addition of 9 g of aluminum isopropoxide (44.0 mmol) and 10 mL benzyl alcohol (96.75 mmol). The resulting mixture was refluxed (65° C.) with constant vigorous stirring for 2 hours. Then solvent was removed on a rotor evaporator (40° C.; 100 mbar), the resulting pale yellow solid mass was transferred into a 250 mL flask containing 130 mL of xylenes preheated at 70° C., and stirred well until complete dissolution. Under constant stirring, aqueous NaOH (0.6 g dissolved in 2 mL water) was added to this solution through a dropping funnel, and boiled at about 130° C. for 1 hour. It was then filtered off when cold, washed with cold xylenes, and the residue was dried under vacuum at 50° C. for 2 hours. The dried pale yellow solid mixture was taken into a 330 mL beaker, followed by the addition of 200 mL aqueous acetic acid (10%). Then it was homogenized in a homogenizer (8000 rpm, 10 minutes), filtered off, several times washed with cold water, and finally the residue was dried in a vacuum oven (50° C.; 350 mbar) for 2 hours. The resulting off-white dried residue was taken into a 300 mL beaker containing 100 mL THF, well homogenized in a homogenizer (8000 rpm, 5 minutes), and filtered off. The filtrate was evaporated off under reduced pressure, and finally the off-white solid product was dried in a vacuum oven (50° C.; 350 mbar; 12 hours) to obtain 4.85 g (10.6 mmol) 3-β-Betulinic Acid of at least about 97 percent purity. Conversion is almost 100 percent and yield is about 96.3 molar percent.

Obviously, numerous modifications and variations of the present invention are possible in light of the above teachings. It is therefore to be understood that within the scope of the appended claims, the invention may be practiced otherwise than as specifically described herein.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A method for preparing a compound of formula (I): 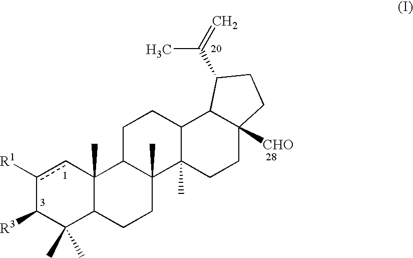
wherein
R¹ is hydrogen or hydroxy;
R³ is acyloxy or oxo (═O); and
the bond shown as ----- is present or absent;
the method comprising contacting a metal alcoholate, a compound of the formula (A): 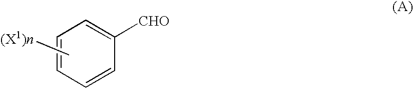
wherein,
each X¹ is independently halo, nitro, hydroxyl, (C₁-C₆)alkyl, (C₁-C₆)alkoxy, COOR¹⁷, or NR⁶R⁷, wherein each of R¹⁶ and R¹⁷ are independently H or (C₁-C₆)alkyl; and
n is 0, 1, 2, 3, 4 or 5;
and a compound of formula (II): 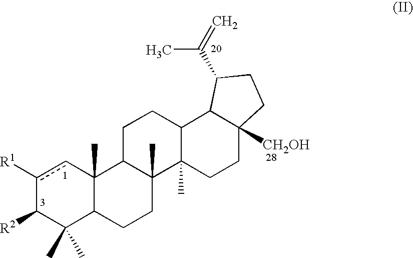
wherein,
R² is hydroxyl or acyloxy;
for a period of time effective to provide the compound of formula (I).

2. The method of claim 1 wherein R¹ is hydrogen.

3. The method of claim 1 wherein R³ is acyloxy.

4. The method of claim 1 wherein R³ is acetoxy.

5. The method of claim 1 wherein R³ is oxo (═O).

6. The method of claim 1 wherein X¹ is chlorine or nitro.

7. The method of claim 1 wherein n is 1.

8. The method of claim 1 wherein the compound of formula (A) is 2-chlorobenzaldehyde or 2-nitrobenzaldehyde.

9. The method of claim 1 wherein the metal alcoholate is an aluminum alcoholate.

10. The method of claim 1 wherein the metal alcoholate is aluminum iso-propoxide [Al(i-OPr)₃].

11. The method of claim 1 wherein R² is hydroxyl.

12. The method of claim 1 wherein R² is acyloxy.

13. The method of claim 1 wherein R² is acetoxy (CH₃C(═O)O).

14. The method of claim 1, wherein the contacting occurs in the presence of a solvent.

15. The method of claim 1, wherein the contacting occurs in the presence of a polar non-protic solvent.

16. The method of claim 1, wherein the contacting occurs at a temperature of at least 20° C.

17. The method of claim 1, having a yield of at least 60 molar percent.

18. The method of claim 1, having a purity of at least 90 percent, as determined by HPLC.

19. The method of claim 1, further comprising purifying the compound of formula (I).

20. The method of claim 1, wherein the metal alcoholate is employed in at least about 2 molar equivalents, in relation to the compound of formula (II).

21. The method of claim 1, wherein the metal alcoholate is employed in at least about 4 molar equivalents, in relation to the compound of formula (II).

22. The method of claim 1, wherein the contacting occurs for at least about 2 hours.

23. The method of claim 1, wherein the compound of formula (II) is employed in at least about 1 kilogram.

24. The method of claim 1, wherein at least about 1 kilogram of the compound of formula (I) is obtained.

25. The method of claim 1, further comprising contacting the compound of formula (I) with an effective amount of an alkali metal chlorite, for a period of time effective to provide a compound of formula (III): 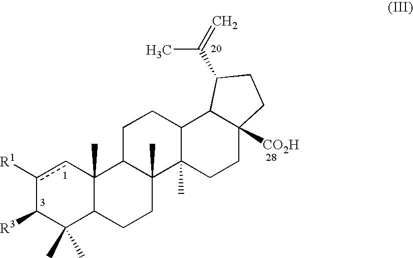
or a salt thereof,
wherein
R¹ is hydrogen or hydroxy; and
R³ is acyloxy or oxo (═O).

26. The method of claim 25 wherein the alkali metal chlorite is NaClO₂, KClO₂, or a combination thereof.

27. The method of claim 25 wherein the compound of formula (I) is contacted with about 5 molar equivalents to about 10 molar equivalents of the alkali metal chlorite, relative to the compound of formula (I).

28. The method of claim 25 wherein the compound of formula (I) is contacted with about 2 molar equivalents to about 5 molar equivalents of the alkali metal chlorite, relative to the compound of formula (I).

29. The method of claim 25, wherein the contacting is carried out at a temperature of about 10° C. to about 120° C.

30. The method of claim 25, wherein the contacting is carried out for a period of time of about 30 minutes to about 48 hours.

31. The method of claim 25, wherein the contacting is carried out in a solvent system selected from water, an alcohol, unsaturated hydrocarbons, mineral oil, ether, dioxane, DMF, DMA, DMSO, benzene, toluene, xylene, pyridine, chloroform, methylene chloride, morpholine, N-methylmorpholine, cyclohexane, cyclohexanone, acetone, ethyl acetate, pyrrole, and pyrrolidone, or a combination thereof.

32. The method of claim 25, wherein at least about 1 kg of the compound of formula (III) is obtained.

33. The method of claim 25, wherein at least about 85 mol % of the compound of formula (III) is obtained, based upon the compound of formula (I).

34. The method of claim 25, further comprising purifying the compound of formula (III).

35. The method of claim 25, further comprising washing the compound of formula (III).

36. The method of claim 25, further comprising recrystallizing the compound of formula (III).

37. The method of claim 25, further comprising separating the compound of formula (III) from any unreacted triterpene compound by converting the compound of formula (III) into a carboxylic acid salt and separating the carboxylic acid salt from the unreacted triterpene compound.

38. The method of claim 37 wherein the carboxylic acid salt comprises a Li, Na, K, Mg, Ca, Sr, Ba, or Al cation.

39. The method of claim 25, wherein the compound of formula (III) is obtained having a purity of at least about 95 wt. %.

40. The method of claim 25, further comprising a free halogen scavenger.

41. The method of claim 25, further comprising a halogen scavenger that is an unsaturated hydrocarbon.

42. The method of claim 25, further comprising a halogen scavenger selected from the group of amylene, cyclohexene, methylcyclohexene and cyclopentene.

43. The method of claim 25, further comprising contacting a metal alcoholate and the compound of formula (III) for a period of time effective to provide a compound of formula (IV): 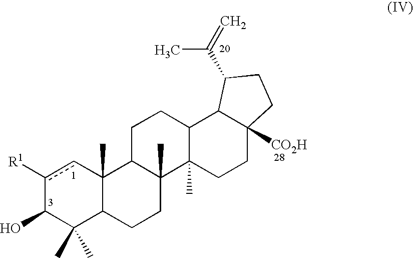
or a salt thereof,
wherein R¹ is hydrogen or hydroxy.

44. The method of claim 43 wherein the bond between carbons 1 and 2 is a single bond.

45. The method of claim 43 wherein R¹ is hydrogen.

46. The method of claim 43 wherein the compound of formula (III) is betulonic acid.

47. The method of claim 43 wherein R³ of the compound of formula (III) is acyloxy.

48. The method of claim 43, wherein the metal alcoholate is aluminum iso-propoxide.

49. The method of claim 43, wherein the contacting occurs in the presence of a compound of formula (B): 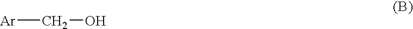
wherein Ar is aryl or heteroaryl.

50. The method of claim 43, wherein the contacting occurs in the presence of a compound of the formula (C): 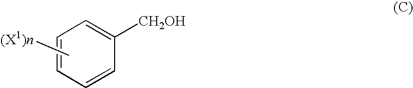
wherein
each X¹ is independently halo, nitro, hydroxyl, (C₁-C₆)alkyl, (C₁-C₆)alkoxy, COOR⁷, or NR¹⁶R¹⁷,
each R¹⁶ and R¹⁷ is independently H or (C₁-C₆)alkyl; and
n is 0, 1, 2, 3, 4 or 5.

51. The method of claim 50 wherein n is 0.

52. The method of claim 50 wherein n is 1.

53. The method of claim 43, wherein the contacting occurs in the presence of a compound of the formula (D): 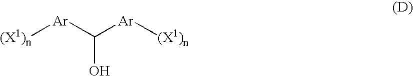
wherein
each Ar is independently aryl or heteroaryl;
each X¹ is independently halo, nitro, hydroxyl, (C₁-C₆)alkyl, (C₁-C₆)alkoxy, COOR¹⁷, or NR¹⁶R¹⁷,
each R¹⁶ and R¹⁷ is independently H or (C₁-C₆)alkyl; and
each n is independently 0, 1, 2, 3, 4 or 5.

54. The method of claim 53 wherein each n is 0.

55. The method of claim 53 wherein each n is 1.

56. The method of claim 53 wherein the compound of formula (D) is a compound of formula (D-I):

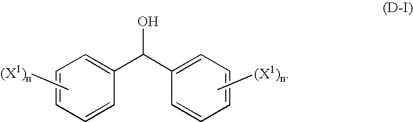

57. The method of claim 53 wherein each n is 0.

58. The method of claim 53 wherein each n is 1.

59. The method of claim 43, wherein the contacting occurs in the presence of benzyl alcohol.

60. The method of claim 43, wherein the contacting occurs in the presence of a solvent.

61. The method of claim 43, wherein the contacting occurs in the presence of a solvent selected from the group of ethyl ether, tetrahydrofuran (THF), dioxane, acetonitrile, dimethylformamide (DMF), dimethylacetamide (DMA), ethyl acetate, or a combination thereof.

62. The method of claim 43, wherein the contacting occurs at a temperature of at least about 50° C.

63. The method of claim 43, further comprising purifying the compound of formula (IV).

64. The method of claim 43, further comprising purifying the compound of formula (IV) by washing the compound of formula (IV).

65. The method of claim 43, further comprising purifying the compound of formula (IV) by washing the compound of formula (IV) with an aqueous acid, an aqueous base, a non-polar aprotic solvent, a polar aprotic solvent, or a mixture thereof.

66. The method of claim 43, wherein the compound of formula (IV) is provided in a yield of at least 95 molar percent.

67. The method of claim 43, wherein the compound of formula (IV) is provided with a purity of at least 95 percent, as determined by HPLC.

68. The method of claim 43, wherein the metal alcoholate is employed in at least about 2 molar equivalents, in relation to the compound of formula (III).

69. The method of claim 43, wherein the metal alcoholate is employed in at least about 4 molar equivalents, in relation to the compound of formula (III).

70. The method of claim 43, wherein the contacting occurs for at least about 2 hours.

71. The method of claim 43, wherein the compound of formula (III) is employed in at least about 1 kilogram.

72. The method of claim 43, wherein at least about 1 kilogram of the compound of formula (IV) is obtained.

73. A method for preparing a compound of formula (IV): 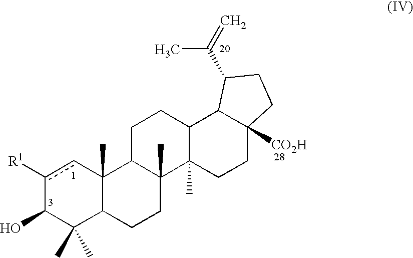
or a salt thereof,
wherein
R¹ is hydrogen or hydroxy; and
the bond shown as ----- is present or absent;
the method comprising the steps of:
(1) contacting a metal alcoholate, a compound of the formula (A): 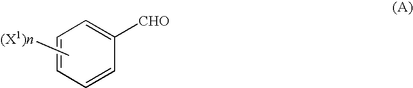
wherein,
each X¹ is independently halo, nitro, hydroxyl, (C₁-C₆)alkyl, (C₁-C₆)alkoxy, COOR¹⁷, or NR⁶R⁷, wherein each of R¹⁶ and R¹⁷ are independently H or (C₁-C₆)alkyl; and
n is 0, 1, 2, 3, 4 or 5;
and a compound of formula (II): 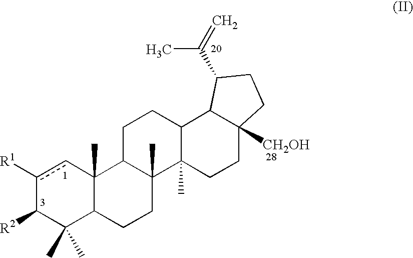
wherein,
R² is hydroxyl or acyloxy;
for a period of time effective to provide the compound of formula (I): 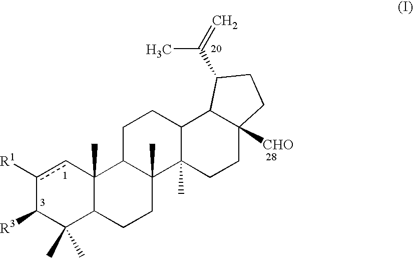
wherein
R¹ is hydrogen or hydroxy;
R³ is acyloxy or oxo (═O);
(2) contacting the compound of formula (I) with an effective amount of an alkali metal chlorite, for a period of time effective to provide a compound of formula (III): 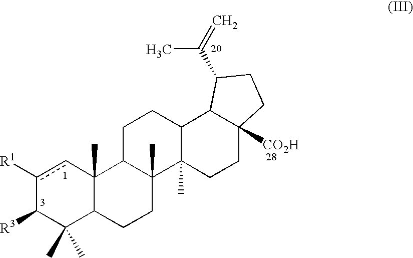
or a salt thereof,
wherein
R¹ is hydrogen or hydroxy;
R³ is acyloxy or oxo (═O); and
(3) contacting a metal alcoholate and the compound of formula (III) for a period of time effective to provide the compound of formula (IV).
